(19) United States
(12) Patent Application Publication
(10) Pub. No.: US 2025/0319104 A1
(43) Pub. Date: Oct. 16, 2025

(54) MOISTURIZING CREAM FOR RADIATION DERMATITIS

(71) Applicant: Arthur W Perry, New York, NY (US)

(72) Inventor: Arthur W Perry, New York, NY (US)

(21) Appl. No.: 18/999,718

(22) Filed: Dec. 23, 2024

Related U.S. Application Data
(60) Provisional application No. 63/615,858, filed on Dec. 29, 2023.

(51) Int. Cl. A61K 31/573; A61K 9/06; A61K 31/352; A61K 31/357; A61K 47/10; A61K 47/14; A61K 47/18; A61K 47/22; A61K 47/24; A61K 47/36; A61K 47/44

(52) U.S. Cl. CPC A61K 31/573; A61K 9/06; A61K 31/352; A61K 31/357; A61K 47/10; A61K 47/14; A61K 47/18; A61K 47/22; A61K 47/24; A61K 47/36; A61K 47/44

(57) ABSTRACT

A topical formulation is provided for treating systems of radiation treatments that includes a moisturizer base, and a plurality of active ingredients that include a corticosteroid, silymarin, and epigallocatechin-3-gallate (EGCG).

### RELATED APPLICATIONS

This application claims priority to U.S. Provisional Patent Application No. 63/615,858 filed Dec. 29, 2023, entitled “Moisturizing Cream for Radiation Dermatitis,” which is hereby incorporated by reference in its entirety.

### FIELD OF THE INVENTION

The present application relates to skin preserving topical compositions. More particularly, the present application relates to formulations that includes a corticosteroid, such as hydrocortisone, silymarin, and/or catechin, such as epigallocatechin-3-gallate (EGCG), in a moisturizer cream that lessens the chance of radiation dermatitis during or as a result of radiation therapy.

### BACKGROUND OF THE INVENTION

Along with surgery and chemotherapy, Radiation Therapy (RT) is a standard treatment for many types of cancer. According to the American Cancer Society, there are nearly 2 million new cases of cancer each year in the United States and 14 million globally. In the United States, there are nearly 4 million people who have undergone RT, nearly a third of all cancer patients. Worldwide, there are approximately 7 million people annually who undergo RT. Radiation injury (acute radiation dermatitis) is a common problem resulting from radiation therapy.

More than 95% of patients who undergo radiation therapy sustain some degree of radiation dermatitis, 85% of patients who undergo RT sustain moderate to severe skin reactions characterized by erythema (redness), edema (swelling), dry and moist (skin shedding (desquamation). Over 30% of patients develop tender weeping skin that sometimes blisters, bleeds, and crusts. Some of these patients progress to full thickness skin loss. These symptoms intensify with each day of radiation exposure and can not only injure a patient's quality of life but can cause a pause in the sequence of radiation therapy. This pause can adversely affect the cancer destroying effects of radiation.

The toxicity of radiation to the skin depends on the daily radiation dose, the cumulative radiation dose, the pre-existing quality of the skin barrier, and the patient's overall health. The toxic effects of RT become apparent within hours to weeks after exposure and are caused by radiation destroying double stranded DNA and inhibiting the cell's ability to divide. This damage is required to achieve a beneficial anti-cancer effect but is considered “collateral damage” when inflicted on the skin that it must pass through to target the cancer. RT causes skin damage by damaging DNA and causing the generation of reactive oxygen species, decreasing skin stem cells, initiating an inflammatory response, and ultimately causing skin cell death. The cumulative effect of this damage results in the classic response known as “radiation dermatitis (RD)”.

Prevention of clinical treatment of radiation dermatitis is currently haphazardly advised by radiation oncologists. Most physicians advise skin washing with mild soap and the application of nonspecific moisturizers to the skin. In patients considered to be at high risk for RD, prophylactic use of steroid cream is recommended. Treatment of existing radiation dermatitis is equally not standardized, with the use of over-the-counter steroid creams being the most common intervention. Bacterial and fungal skin infections are common, and often require the temporary or even permanent cessation of RT. Interruptions may decrease RT effectiveness. Accordingly, there is a need for formulations for use in lessening the chance of and/or treating radiation dermatitis.

### SUMMARY OF THE INVENTION

In one embodiment, a topical cream/gel/lotion is provided that has a pH in the range of 5-8, which includes a moisturizer base, with ceramides, emollients, humectants, and occlusives designed to maintain the barrier function of the skin throughout the RT process, and at least one of a corticosteroid, silymarin and epigallocatechin-3-gallate (EGCG).

In one embodiment, the topical cream/gel/lotion/liquid includes from about 0.1% to about 5%, or more preferably about 1% hydrocortisone, or another stronger corticosteroid ranging in concentration from 0.01% to 1%, the corticosteroid selected from the group consisting of aclometasone, diproprionate, amcinonide, augmented betamethasone dipropionate betamethasone valerate, betamethasone diproprionate, clobetasol proprionate, clocortolone pivalate, desonide, desoximetasone, diflorasone, diacetate, fluandrenolide, fluocinolone acetonide, fluocinonide, fluticasone, propionate, halcinonide, halobestasol, propionate, hydrocortisone butyrate, methyl prednisolone, mometasone furoate, prednicarbate, and triamcinolone acetonide. Percentages refer to weight percent unless specified otherwise.

Each of the key ingredients in this application have been shown to decrease the severity of RD. It is expected that the combined effect of these ingredients, as disclosed herein, will allow unprecedented tolerability of RT.

In another aspect, a metered system is provided to coat the skin with the topical cream/gel/lotion/liquid prior to RT treatment which remains in place for 0.5 to 4 hours, or preferably at least 2 hours under a supplied occlusive dressing. The system may be applied periodically, e.g., 5 to 16 hours, preferably at least every 12 hours for the duration of the RT. After a 4 day “drug holiday,” application of the topical cream/gel/lotion/liquid may be resumed/continued for an additional 3 to 8 weeks, or preferably at least 6 weeks.

The use of this topical cream/gel/lotion/liquid disclosed herein will decrease the onset of radiation induced skin irritation (RD) and decrease its severity once it occurs. The topical cream/gel/lotion/liquid will decrease itching, pain, redness, and wound breakdowns.

The use of this topical cream/gel/lotion/liquid will help consumers tolerate radiation therapy and make it more likely that they will complete the entry cycle of RT, thereby achieving therapeutic goals that will improve the likelihood of curing the cancer.

This invention is unique in that there is no commercially available compositions designed to prevent radiation dermatitis that achieve the unexpected benefits of the combined moisturizer with corticosteroids, ECGC, and silymarin, as disclosed herein.

### DETAILED DESCRIPTION OF THE PREFERRED EMBODIMENTS

Prevention of RD is a reasonable goal during the administration of RT. Prevention involves maintaining a proper “barrier function” of the skin, minimizing or eliminating the deleterious action of RT on the skin and boosting the skin's reparative abilities. Prevention may begin with the use of mild soap to wash the skin, followed by the application of the novel moisturizing topical cream/gel/lotion/liquid (hereinafter referred generally to a formulation(s)) disclosed herein.

In this regard, the present application provides an exceptionally effective skin moisturizer that includes a moisturizer base, which incorporates three ingredients that are believed be the most efficacious in the prevention of RD. Generally, the moisturizer base includes a mixture of emollients, humectants, ceramides, and occlusives—with a composition/characteristics similar to that of normal skin, e.g., water content, fat content, etc.

Such moisturizers prevent skin barrier disruption and are helpful when the epidermal barrier is breached. They are important in low humidity situations and in diseases, or after the use of barrier disrupters, such as cleansers, astringents, topical medicines, and radiation exposure.

The epidermal skin barrier is made of proteins from dead cells plastered together. Under the epidermis cells are held together by layers of fats. If either layer is breached, increased water loss results. The skin then appears dry, scaly, rough, and it can crack and itch. The stratum corneum, the upper layer of skin, should contain 20-35% water. When the level drops below 10% water content, dry skin changes occur.

In one embodiment, the formulation(s) disclosed herein includes an optimal or optimized moisturizer base with at least one and preferably at least three lipid components: ceramides, cholesterol, and/or free fatty acids. In one embodiment, the moisturizer base contains occlusives that slow water loss by forming a hydrophobic film on skin between the skin cells, humectants that attract water from the underlying dermis and humid air and deliver it to the stratum corneum of the epidermis, and/or emollients that fill in tiny spaces between skin cells and make the skin feel smooth. Humectants, such as hyaluronic acid, increase water content of the skin, which decrease the risk of RD. Emollients, constituents of moisturizers, such as olive and jojoba oils, reduce radiation dermatitis.

The moisturizer base generally replaces the natural oils that are washed away by soaps and degraded by chemicals during radiation therapy. The novel formulation may also replenish the skin barrier, keeping skin healthy and attractive.

The ceramides are important for the maintenance of the skin barrier, and thus skin health. The base, according to at least one embodiment, contains hyaluronic acid, important for maintaining moisture in the skin. The base may also contain jojoba, olive and/or safflower oils and/or olive derived squalene oils that are beneficial to the skin along with Vitamins E and B5 (Panthenol), vitamins that are important for good skin health. In one embodiment, the ceramide includes Ceramide 2, by Sederma, Inc.

The formulation, according to at least one embodiment, contains topical corticosteroids which prevent the incidence of and treating severe RT. Topical corticosteroids have anti-inflammatory effects and can inhibit radiation induced cytokines. Both mild and potent steroids can prevent or delay severe RT. Specifically, 0.1% to about 5%, or preferably about 1% hydrocortisone may be used in delaying the onset of RT. A variety of other corticosteroids in similar concentrations may be used.

The formulation according to at least one embodiment of the application contains silymarin (milk thistle) in a 0.1% to about 5%, or preferably about 1% concentration. This has been shown to stall the onset of RT symptoms and decrease their severity. The formulation according to at least one embodiment contains epigallocatechin-3 Gallate (EGCG) in a 0.01% to about 0.1% concentration. ECGC is a catechin, a phenolic compound that occurs naturally in cocoa, tea, and various berries. Catechins are antioxidants that scavenge free radicals and other toxins that have been shown to decrease the severity of RT and control burning, erythema (redness), itching and pain from RT. A dose of 660-2574 umol/L is recommended.

In one embodiment, skin cream is provided that is designed to protect skin during radiation therapy procedures, keeping the water content optimal and minimizing free radical damage that typically occurs during radiation exposure. It incorporates steroids, silymarin and epigallocatechin-3-gallate (EGCG) into a comprehensive moisturizer base.

In one embodiment, the base includes occlusive ingredients, such as cyclomethicone, polyunsaturated lipids such as squalene, and/or phospholipids such as lecithin. These ingredients slow water loss by forming a hydrophobic film on skin between skin cells.

In one embodiment, the humectants include at least one of glycerin, mucopolysaccharides, such as hyaluronic acid, and/or panthenol. These attract water from the underlying dermis and humid air and deliver it to the stratum corneum of the epidermis. Hyaluronic acid has been shown to decrease pain and RD.

In one embodiment, the emollients include at least one of jojoba oil, dimethicone, olive oil and saturated fatty alcohols, such as cetyl alcohol or stearyl alcohol. These fill in tiny spaces between skin cells and make the skin feel smooth.

In one embodiment, the formulation contains corticosteroids. These have anti-inflammatory properties by inhibiting the surgery of radiation induced cytokines. The formulation may include hydrocortisone from about 0.1% to about 5% concentrations. The amount of a more potent corticosteroid may range in concentration from 0.01% and 1%, such as aclometasone and diproprionate, amcinonide, augmented betamethasone dipropionate betamethasone valerate, betamethasone dipropionate, clobetasol proprionate, clocortolone pivalate, desonide, desoximetasone, diflorasone diacetate, flurandrenolide, fluocinolone acetonide, fluocinonide, fluticasone propionate, halcinonide, halobetasol propionate, hydrocortisone butyrate, methyl prednisolone, mometasone furoate, prednicarbate, and/or triamcinolone acetonide.

In one embodiment, the formulation contains about 0.1% to about 10%, or preferably 1% silymarin (Silybum marianum). Silymarin, also known as milk thistle, delayed the occurrence of RD and reduced severity. The radioprotective effects are due to its antioxidant, anti-apoptosis, and anti-inflammatory properties.

In one embodiment, the formulation contains epigallocatechin-3-gallate (EGCG) 0.01% to about 0.1%, or preferably 0.03%, with a range of concentration between 660-2574 umol/L. This phenolic antioxidant is found in several plants such as green and black tea. It inhibits cellular oxidation and prevents free radical damage to cells. EGCG has been shown to decrease radiation dermatitis and reduce pain, burning, and itching during and after RT.

In one embodiment the moisturizer base is composed of the following components by weight percent:
Water at least 20%, preferably 20-35%
Lecithin (phosphatidylcholine) 0.1%-5%
Phytosphingosine 0.1% to 1%
Cholesterol 0.1%-1%
Glycerin 1%-5%
Olive oil 1%-5%
Safflower oil 1%-5%
Squalene (olive) 1%-5%
Sodium Hyaluronate 0.1%-5%
Jojoba Oil 1%-5%
Ceramide 2-0.1%-1%
Panthenol 0.1%-1%
DL alpha-tocopherol in soybean oil 0.1%-1%1
PPG-3 Benzyl Ether Myristate 1-5%
Sodium Steroyl Glutamate 0.5%-5%
Cetearyl Alcohol and Cetearyl Glucoside 0.1%-5%
Cetyl Alcohol 0.1% c-2%
Arachidyl Alcohol, Behenyl Alcohol, Arachidyl glucoside 1%-5%
Dicaprylyl Carbonate 1%-10%
Xanthan Gum 0.1%-1%

The product may be preserved with:
Phenoxyethanol
Ethylhexylglycerin
Benzyl Alcohol

The active ingredients according to the preferred embodiment are:
EGCG 95% Epigallocatechin Gallatyl Glucoside 0.03%
Hydrocortisone 1%
Silymarin Silybum Marianum Extract 1%

The mixture of the base is made as follows. The following components comprise Phase A: Cetyl Alcohol, Montanov 68, Montanov 202, ceramide 2, Crodamol STS, ECGC, Cetiol CC, cholesterol, lecithin, phytosphingosine. The process may begin by combining and/or mixing Phase A components in a vessel and heating them to 80 C.

The following components comprise Phase B: Water, Eumulgin SG, D-panthenol, hyaluronic acid, benzyl alcohol and Euxyl PE, which are heated to 80 C. Each of the heated Phase B ingredients is added to a vessel one at a time and mixed to dissolve. Using a propeller under high shear xanthan gum is added to the Phase B solution and mixed for 20 minutes.

Phase A is then be combined with Phase B under a Silverson Type homogenizer for 5 minutes, then switched to a sweep mixer under slow agitation to cool down. When temperature reaches room temperature, Phase C ingredients may then be added to the Phase A and B mixture. Phase C ingredients includes a mixture of water, coco caprylate, safflower seed oil, olive fruit oil, jojoba oil, cetearyl alcohol, glycerin, xanthan gum, benzyl alcohol, phenoxyethanol, and ethylhexylglycerin. The ABC mixture is then cooled to 50° C.

Phase D ingredients include a mixture of tocopherol and soybean oil which is added to the cooled ABC mixture and mixed for 5 minutes. Phase E mixture includes Silymarin, glycerin and water, which is mixed to ABCD when ABCD cools to 35° C., phase E is mixed in until smooth, eventually cooling the final mixture to at 25° C.

### Packaging

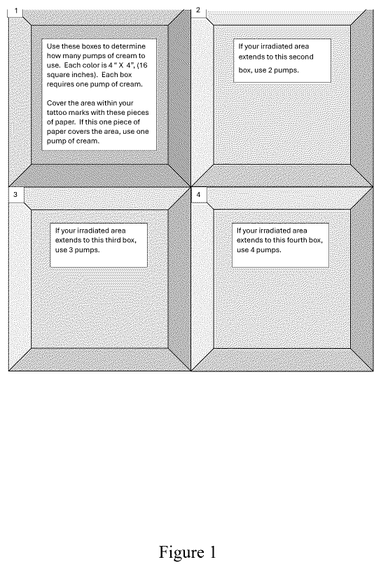

The room temperature cream may then be loaded into airless pumps, with the pump metered appropriately to deliver a range of 1-1.5 ml of cream per pump. Each pump is designed to deliver the amount of cream necessary to treat a 4″×4″ square area (16 square inches) of skin. The consumer will be directed to measure the area within the tattooed radiation zone and select the appropriate number of cream pumps to apply. A graphic will preferably accompany the cream to show how to measure and how many pumps to use, as shown in FIG. 1.

In one embodiment, a consumer kit is provided that will include 1 bottle of the skin cream, an instruction booklet with usage guidance, (4) 4′×4″ paper templates and 28 (a 14-day supply) commercially available pieces of 5″×9″ petrolatum impregnated fine mesh gauze. The template may be folded twice over to a 4″×4″ size. The cream is applied beginning the night before RT begins and is reapplied twice a day—prior to and after the daily RT sessions, for the duration of the RT. The area will then be gently washed off and the process is repeated preferably every 12 hours. The cream may be applied beginning the night before RT begins and continues for the duration of the RT. At the conclusion of the RT, there is a 4 day “holiday” where no cream is used. The cream is then resumed twice daily, preferably for an additional 6 weeks.

What is claimed is:

1. A topical formulation having a pH in the range of 5-8, comprising a moisturizer base, and a plurality of active ingredients comprising a corticosteroid, silymarin, and epigallocatechin-3-gallate (EGCG).

2. The topical formulation of claim 1, wherein the corticosteroid comprises 0.1 to 1 weight percent hydrocortisone.

3. The topical formulation of claim 1, wherein the corticosteroid comprises at least one selected from the group consisting of aclometasone, diproprionate, amcinonide, augmented betamethasone dipropionate betamethasone valerate, betamethasone diproprionate, clobetasol proprionate, clocortolone pivalate, desonide, desoximetasone, diflorasone, diacetate, fluandrenolide, fluocinolone acetonide, fluocinonide, fluticasone, propionate, halcinonide, halobestasol, propionate, hydrocortisone butyrate, methyl prednisolone, mometasone furoate, prednicarbate, and triamcinolone acetonide and wherein a concentration of the corticosteroid is 0.01 to 1 weight percent.

4. The topical formulation of claim 1, wherein the moisturizer base comprises a mixture of an emollient, a humectant, a ceramide, and an occlusive.

5. The topical formulation of claim 4, wherein the moisturizer base comprises at least three lipid components.

6. The topical formulation of claim 5, wherein the at least three lipid components comprise a ceramide, cholesterol, and a free fatty acid.

7. The topical formulation of claim 4, wherein the humectant comprises at least one of glycerin, a mucopolysaccharide, and panthenol.

8. The topical formulation of claim 4, wherein the emollient comprises at least one of olive oil, jojoba oil, safflower oil, an olive derived squalene oil. dimethicone, and saturated fatty alcohols.

9. The topical formulation of claim 4, wherein the occlusive comprises at least one of cyclomethicone, a polyunsaturated lipid, and a phospholipid.

10. The topical formulation of claim 1, wherein the epigallocatechin-3 Gallate (EGCG) has a concentration of 0.01 to 0.1 weight percent.

11. The topical formulation of claim 1, wherein the silymarin has a concentration of 0.1 to 10 weight percent.

12. The topical formulation of claim 1, wherein the active ingredients comprise a mixture of:
EGCG 95% Epigallocatechin Gallatyl Glucoside 0.03 weight percent,
hydrocortisone, and
silymarin 1 weight percent, the formulation further comprising 0.1 weight percent to 5 weigh percent of each of:
Lecithin (phosphatidylcholine)
Phytosphingosine,
Cholesterol,
Glycerin,
Olive oil,
Safflower oil,
Squalene (olive),
Sodium Hyaluronate,
Jojoba Oil,
Ceramide,
Panthenol,
DL alpha-tocopherol in soybean oil,
PPG-3 Benzyl Ether Myristate,
Sodium Steroyl Glutamate,
Cetearyl Alcohol and Cetearyl Glucoside,
Cetyl Alcohol,
Arachidyl Alcohol, Behenyl Alcohol, Arachidyl glucoside’
Dicaprylyl Carbonate, and
Xanthan Gum.

13. A method for treating symptoms of radiation treatment comprising applying a formulation having a pH in the range of 5-8 to skin topically at a site of interest, the formulation comprising a moisturizer base, and a plurality of active ingredients comprising a corticosteroid, silymarin, and epigallocatechin-3-gallate (EGCG), the method further comprising reapplying the formulation to the skin every 5 to 16 hours during the radiation treatment, and reapplying the formulation to the skin after a 4 day drug holiday.

14. The method of claim 13, wherein the formulation is applied 3 to 8 weeks after the 4 day drug holiday.

15. The method of claim 13, wherein the formulation is applied under an occlusive covering.

16. A method for manufacturing the formulation for treating symptoms of radiation treatment comprising:
mixing Cetyl Alcohol, Montanov 68, Montanov 202, ceramide 2, Crodamol STS, ECGC, Cetiol CC, cholesterol, lecithin, and phytosphingosine in a vessel;
mixing water, Eumulgin SG, D-panthenol, hyaluronic acid, benzyl alcohol and Euxyl PE in a second vessel one at a time until dissolved, and adding xanthan gum under high shear;
combining content of the first and second vessel;
mixing water, coco caprylate, safflower seed oil, olive fruit oil, jojoba oil, cetearyl alcohol, glycerin, xanthan gum, benzyl alcohol, phenoxyethanol, and ethylhexylglycerin in a third vessel, and adding content of the third vessel to the combined first and second vessel;
mixing tocopherol and soybean oil in a fourth vessel, and combining content of the fourth vessel with the combined first, second, and third vessel; and
mixing silymarin, glycerin, and water in a fifth vessel and combining the content of the fifth vessel with the combined first, second, third, and fourth vessel to form a final mixture.

17. A method of claim 16, comprising loading the final mixture into an airless pump that delivers a range of 1-1.5 ml of cream per pump.
